(19) United States
(12) Patent Application Publication
(10) Pub. No.: US 2008/0021456 A1
(43) Pub. Date: Jan. 24, 2008

(54) SACRAL OR ILIAC CROSS CONNECTOR

(75) Inventors: Munish Gupta, Carmichael, CA (US); Nam T. Chao, Marlborough, MA (US); Ross Sylvia, Taunton, MA (US)

Correspondence Address: NUTTER MCCLENNEN & FISH LLP, WORLD TRADE CENTER WEST, 155 SEAPORT BOULEVARD, BOSTON, MA, 02210-2604, US

(73) Assignee: DEPUY SPINE, INC., Raynham, MA (US)

(21) Appl. No.: 11/459,176

(22) Filed: Jul. 21, 2006

(51) Int. Cl. A61F 2/30

(52) U.S. Cl. 606/61

(57) ABSTRACT

Implantable devices and methods for correcting spinal deformities or degeneration are disclosed. The devices and methods have particular application in the spinopelvic region and on the sacrum or ilium. In one embodiment, a cross connector is provided and can include a cross member with a connector head slidably disposed thereon. The cross member can be adapted for attachment to one or more anchoring elements, and the sliding connector head can include a first receiving portion defined by an opening through the connector head for receiving the cross member, and a second receiving portion defined by a recess between first and second opposed arms for receiving a spinal fixation element, such as a spinal rod. A guide channel can optionally be provided on the cross member for guiding the movement of the sliding connector head. In other embodiments, the device can include a locking mechanism for locking a spinal fixation element within the connector head, and for securing the connector head in place on the cross member.

### FIELD OF THE INVENTION

The present invention relates to implantable devices and methods for correcting spinal deformities or degeneration, and for connecting a spinal fixation element to bone.

### BACKGROUND OF THE INVENTION

Spinal deformities, which include rotation, angulation, and/or curvature of the spine, can result from various disorders, including, for example, scoliosis (abnormal curvature in the coronal plane of the spine), kyphosis (backward curvature of the spine), and spondylolisthesis (forward displacement of a lumbar vertebra). Other causes of an abnormally shaped spine include trauma and spinal degeneration with advancing age. Early techniques for correcting such deformities utilized external devices that applied force to the spine in an attempt to reposition the vertebrae. These devices, however, resulted in severe restriction and in some cases immobility of the patient. Furthermore, current external braces have limited ability to correct the deformed spine and typically only prevent progression of the deformity. Thus, to avoid this need, doctors developed several internal fixation techniques to span across multiple vertebrae and force the spine into a desired orientation.

To fix the spine, surgeons attach one or more fixation elements (typically rods or plates) to the spine at several fixation sites, typically in the lumbar and sacral region, to correct and stabilize the spinal deformity, prevent reoccurrence of the spinal deformity, and stabilize weakness in trunks that results from degenerative discs and joint disease, deficient posterior elements, spinal fracture, and other debilitating problems. Where rods are used, they may be pre-curved or curved intraoperatively to a desired adjusted spinal curvature. Wires as well as bone screws or hooks can be used to pull individual vertebra or bone structure toward the rod, thereby anchoring the device to bone. The procedure may also include fusion of the instrumented spinal segments.

Once anchored, the rod-based systems are under stress and subjected to significant forces, known as cantilever pullout forces. As a result, surgeons are always concerned about the possibility of the implant loosening or the bone screws pulling out of the bone, especially where the system is anchored to the sacrum or ilium. The sacrum and ilium are usually of poor bone quality, consisting primarily of cancellous bone with thin cortical bone, magnifying the problem when fixation elements must be fixed to them. Thus, surgeons generally seek to attach implants in the most secure and stable fashion possible while at the same time addressing a patient's specific anatomy. While several current techniques exists for anchoring fixation elements to the sacrum and ilium, the current techniques require precise contouring and placement of spinal rods on the sacrum and/or ilium during surgery. The task becomes more difficult when, as is often called for, a surgeon must construct a framework of articulated spinal rods. As a result, while several different rod-based systems have been developed, they can be cumbersome, requiring complicated surgical procedures with long operating times to achieve correction. Furthermore, intraoperative adjustment of rod-based systems can be difficult and may result in loss of mechanical properties due to multiple bending operations. Surgeons find a number of the current techniques to be complex and challenging to implement.

Accordingly, there is a need in this art for novel implantable devices for correcting spinal deformities or degeneration that reduce the complexity of surgery, are compatible with current surgical techniques, and can be easily and intraoperatively customized.

### SUMMARY OF THE INVENTION

Implantable devices, systems, and methods useful for connecting a spinal fixation element to bone, preferably in the spinopelvic region on the sacrum or ilium, are disclosed. In one embodiment, an implantable device is provided for connecting a spinal fixation element to a patient's sacrum or ilium. The device can include a cross member having opposed first and second ends, and a guide channel formed thereon between the first and second ends. The cross member can optionally include one or more bend zones formed therein. The device can also include a connector head slidably disposed on the guide channel and having a first receiving portion defined by an opening through the connector head for receiving the cross member, and a second receiving portion defined by a recess formed between first and second opposed arms for receiving a spinal fixation element.

In one embodiment, the second receiving portion of the connector head can include a seat disposed in the recess for receiving a spinal fixation element. The seat can be, for example, saddle-shaped. The connector head can also include a guide pin extending from the connector head into the guide channel for guiding movement of the connector head along the cross member. For example, the guide pin can extend from the seat into the guide channel for guiding movement of the connector head. In another embodiment, the opening that defines the first receiving portion of the connector head can have a width that exceeds a width of the cross member such that the connector head can pivot relative to the cross member.

The device can also include a locking mechanism adapted to mate to the connector head to lock a spinal fixation element therein and to lock the connector head in a fixed position relative to the cross member. The connector head can include, for example, screw threads formed thereon for mating with a locking mechanism.

In another embodiment, the device can include a second connector head fixedly or slidably attached to the cross member. The second connector head can include a first receiving portion defined by an opening through the connector head for receiving the cross member, and a second receiving portion defined by a recess formed between first and second opposed arms for receiving a spinal fixation element. A second guide channel can be provided on the cross member between the first and second ends, and the second connector head can be slidably disposed on the second guide channel. A guide pin can optionally extend from the second connector head into the second guide channel for guiding movement of the second connector head along the cross member. The device can also include a second locking mechanism adapted to mate to the second connector head to lock a spinal fixation element therein and thereby lock the second connector head in a fixed position relative to the cross member.

The present invention also provides systems for connecting a spinal fixation element to a patient's sacrum or ilium. In one embodiment, the system can include a cross member having opposed first and second ends, a spinal fixation element, a connector head slidably disposed on the cross member between the first and second ends and adapted to receive the spinal fixation element, and a locking mechanism for locking the spinal fixation element within the connector head to prevent movement of the connector head relative to the cross member. In another embodiment, the system can include at least one anchoring element connected to at least one of the first and second ends of the cross member for anchoring the cross member to bone.

In one exemplary embodiment, the connector head can include a first receiving portion defined by an opening through the connector head for receiving the cross member, and a second receiving portion defined by a recess between first and second opposed arms for receiving a spinal fixation element. The second receiving portion can include a seat disposed in the recess thereof for receiving the spinal fixation element. In another embodiment, the cross member can include a guide channel formed between the first and second ends, and the connector head can be slidably disposed on the guide channel. A guide pin can extend from the seat of the connector head and into the guide channel for guiding the movement of the connector head along the cross member.

In yet another embodiment, the system can include a second spinal fixation element, and a second connector head disposed on the cross member between the first and second ends and adapted to receive the second spinal fixation element. The second connector head can be slidably or fixedly disposed on the cross member.

Exemplary methods for anchoring a spinal fixation element to a patient's sacrum or ilium are also provided, and in one embodiment a method can include anchoring a cross member to bone, sliding a connector head along the cross member, positioning a spinal fixation element within the connector head, and locking the spinal fixation element within the connector head to thereby lock the connector head in a fixed position relative to the cross member. The method can also include positioning a second spinal fixation element within a second connector disposed on the cross member. For example, the second connector can be slid along the cross member to facilitate positioning of the second spinal fixation element therein, and the second spinal fixation element can be locked within the second connector head to thereby lock the second connector head in a fixed position relative to the cross member. In certain exemplary embodiments, each connector head is slid along a guide channel formed in the cross member. The method can also include bending the cross member at a bend zone, and/or pivoting the connector head relative to the cross member.

### BRIEF DESCRIPTION OF THE DRAWINGS

The invention will be more fully understood from the following detailed description taken in conjunction with the accompanying drawings, in which:

FIG. 1 illustrates one exemplary embodiment of a spinal construct having longitudinal spinal fixation rods that are anchored to the ilium;

FIG. 2 is a top view of one exemplary embodiment of a cross connector;

FIG. 3 is a side view of the cross connector shown in FIG. 2;

FIG. 4 is an exploded view of a connector head of the cross connector shown in FIGS. 2 and 3;

FIG. 5 is an assembled view of the connector head shown in FIG. 4;

FIG. 6 is a perspective view of the cross connector shown in FIGS. 2 and 3;

FIG. 7 is a perspective view of another embodiment of a cross connector, shown with connector heads removed from the cross member;

FIG. 8 illustrates one exemplary embodiment of a spinal construct having longitudinal spinal fixation rods that are anchored to the sacrum;

FIG. 9 is a side view of the construct shown in FIG. 8;

FIG. 10 illustrates one exemplary embodiment of a spinal construct having longitudinal spinal fixation rods that are anchored to the sacrum and the ilium; and

FIG. 11 is a side view of the construct shown in FIG. 10.

### DETAILED DESCRIPTION OF THE INVENTION

Certain exemplary embodiments will now be described to provide an overall understanding of the principles of the structure, function, manufacture, and use of the devices and methods disclosed herein. One or more examples of these embodiments are illustrated in the accompanying drawings. Those of ordinary skill in the art will understand that the devices and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention.

The present invention generally provides devices and methods for connecting a spinal fixation element to bone in a patient's spinopelvic region. The devices and methods have particular applicability to fixations to the sacrum or ilium individually, or to both the sacrum and ilium in combination. However, these fixations are only by way of example only, and no particular implant site is necessary. A range of spinal applications, including attachments between vertebrae or on other areas of the pelvis, are possible and will depend on patient anatomy and diagnosis and the desired configuration of the spinal implant.

The devices and methods disclosed herein allow a surgeon to easily and quickly customize an appropriate implantable device for correcting the spine. Rather than requiring precise and difficult contouring of spinal rods, a surgeon will often be able to “drop and lock” the implantable device, resulting in a simpler surgical technique. The devices and methods can be used to implement and improve current surgical techniques, although those of skill in the art will no doubt find many applications for the device and methods disclosed herein, including their use in newly developed techniques.

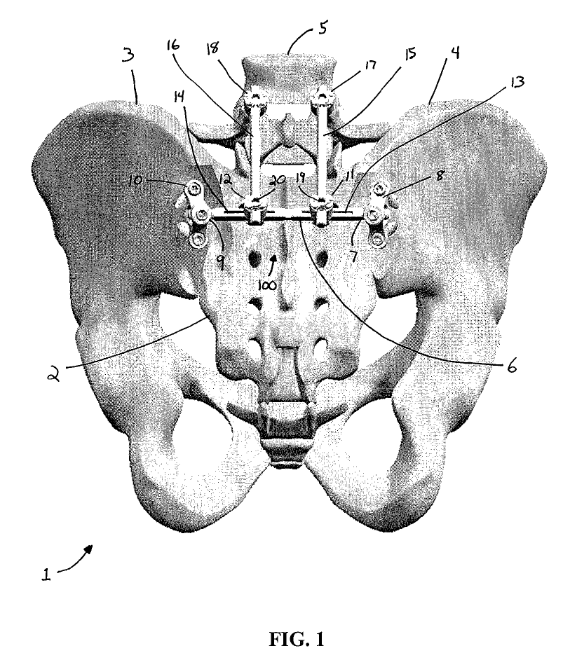

FIG. 1 illustrates one exemplary embodiment of a spinal construct anchored to the ilium. A posterior view of the spinopelvic region 1 shows the sacrum 2, the left ilium 3, and the right ilium 4. The lumbar vertebrae of the spine extend upwards from the sacrum 1 and, for clarity, only the fifth lumbar vertebra 5 is illustrated, although a device could extend farther up the spine or have attachment points elsewhere on the spine.

The general components of the embodiment of FIG. 1 will now be described. It should be noted that FIG. 1 merely serves as an illustration of a possible arrangement of components, and accordingly not all of the components of FIG. 1 may be necessary to achieve the advantages of the invention, nor is the specific geometry of FIG. 1 required.

In general, the construct includes a cross connector 100 having a cross member 6 that extends laterally from the left ilium 3 to the right ilium 4. One end 7 of the cross member 6 is attached to a first anchoring element 8, shown as a bone plate accommodating twin bone screws fastened into the right ilium 4. The opposing end 9 of the cross member 6 is attached to a second anchoring element 10 fastened into the left ilium 3. The first and second anchoring elements 8, 10 each have a receiver head formed thereon and sized to accept an end 7, 9 of the cross member 6, which sits within an opening in the receiver head. A fastener, such as a set screw, bolt or clamp, or the like, can be used to secure the ends 7, 9 of the cross member 6 to each anchoring element 8, 10. The anchoring elements include, for example, the spinal connectors described in more detail in a U.S. patent application filed on even date herewith and entitled “Sacral or Ilium Screw Connector,” by Nam T. Chao et al. (Attorney Docket No. 101896-469), which is hereby incorporated by reference in its entirety. A person skilled in the art will appreciate that various other anchoring elements, such as bones screws, hooks, and the like, can be used to anchor the cross member to bone.

As further shown, two connector heads 11, 12 are disposed on the cross member 6 and are capable of sliding on the cross member 6 along guide channels 13, 14. The construct can also include longitudinal spinal fixation elements, which are in the form of spinal rods 15, 16, that sit in U-shaped recesses in the connector heads 11, 12. The first spinal rod 15 can extend from the connector head 11 to an anchor 17, shown attached to fifth lumbar vertebra. The second spinal rod 16 can extend from the connector head 12 to an anchor 18, also shown attached to the fifth lumbar vertebra. While not shown, the longitudinal spinal rods 15, 16 can connect to other anchors, or to additional cross members, spinal rods, or other types of spinal implants. Either prior to or after placement of the spinal rods 15, 16, into the connector heads, a surgeon may freely move the connector heads 11, 12 along the length of the cross member 6. Thus, the sliding connector heads 11, 12 on the cross member 6 allow a surgeon to easily position the cross member 6 and/or the spinal rods 15, 16 in a desired location. The sliding movement can also facilitate positioning of the spinal rods 15, 16 in the connector heads 11, 12. Because the connector heads 11, 12 can be freely moved, a surgeon may easily customize the configuration of the device before locking it into a final position. This flexibility reduces the need for complex contouring of the cross member 6, spinal rods 15, 16, or other components of the spinal construct.

All of the components described herein, including the cross member 6, the connector heads 11, 12, the anchoring elements 8, 10, and the spinal rods 15, 16, can be made of a bio-compatible material suitable for implantation in the body and capable of withstanding the forces placed upon it during and after implant. Exemplary materials include, by way of non-limiting example, titanium alloy or stainless steel.

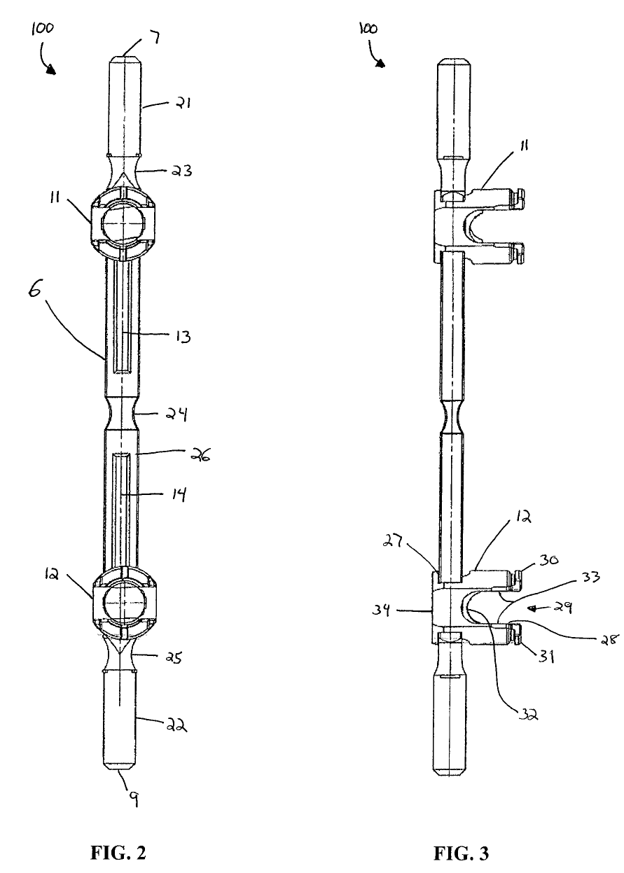

FIGS. 2 and 3 show a top view and a side view, respectively, of the cross connector 100 of FIG. 1. The cross member 6 is primarily composed of an elongate shaft or rod having opposed ends 7, 9. The cross member 6 may have a cross-sectional shape that is circular, square, rectangular, or another shape which allows for the attachment of a connector head. Alternatively, the shape of the cross section may vary along the length of the cross member 6. In the illustrated embodiment, the cross member 6 generally has a rectangular cross-sectional shape along a length thereof. The ends, however, can include anchor attachment portions 21, 22 having a circular cross-section shape that is suitable for attachment to a bone anchoring element, such as the anchoring elements 8, 10 shown in FIG. 1. The size and shape of each attachment portion 21, 22 can be suited to the type of anchoring element used. One of ordinary skill in the art will recognize that a variety of anchoring elements and corresponding anchor attachment portions are possible, as long as an adequate attachment to bone is achieved. Typically the anchor attachment portion is inserted into an opening, such as a hole, slot, U-shaped recess, or the like, on the anchoring element and then secured to the anchoring element with a fastener, such as a set screw, clamp, or bolt. The attachment portion need not be longer than is necessary for insertion into the opening of the anchoring element, however, an attachment portion that is longer than the width of the opening of the selected anchoring element can be advantageous because it allows for some adjustment of position after insertion of the attachment portions 21, 22 of the cross member 6 into the anchoring elements.

As further shown in FIG. 2, the cross member 6 can also optionally include bend zones 23, 24, 25 formed thereon for allowing a surgeon to contour the cross member 6 as desired while still retaining the integrity of the cross member 6. The bend zones can be formed to have a smaller diameter (if the cross member 6 has a cross-sectional shape that is circular) or width (if the cross-member 6 has a cross-sectional shape that is non-circular) than the remainder of the cross member 6, so that when a surgeon applies a bending force to the cross member 6 the bend zone 23, 24, 25, which is weaker due to its smaller size, deflects under the force more easily than the remainder of the cross member 6.

The cross member 6 can also optionally include one or more guide channels formed therein for guiding the movement of the connector head along the cross member 6. FIG. 2 illustrates two guide channels 13, 14 formed in the cross member 6. The first connector head 11 can be adapted to slide along the length of guide channel 13, and the second connector head 12 can be adapted to slide along the length of guide channel 14. The guide channels 13, 14 may take the form of a trench, slot, or cutout in the surface of the cross member 6. The depth of guide channels 13, 14 can also extend all the way through the cross member 6. A guide channel portion 26 of the cross member 6 is shown with a rectangular cross section, although other cross-sectional shapes could be used. Alternatively, the guide channels 13, 14 may be defined by two parallel ridges on the cross member 6. Although FIG. 2 shows two guide channels 13, 14, it is possible to have only one guide channel with one or more connector heads sliding along it (by omitting bend zone 24, for example). More than two guide channels are also possible and may be useful, for example, to accommodate a larger number of connector heads disposed on the cross member 6. Though the interiors of the guide channels 13, 14 are smooth in FIG. 2, they can include serrations or teeth formed therein to facilitate the selection of preset or determinative positions for the connector heads 11, 12. In addition, the connector heads 11, 12 could slide along the length of the cross member 6 alone, without any guide channels.

The connector heads 11, 12 can serve, generally speaking, as connection points or joints between elements of the spinal implant. For example, the connector heads 11, 12 can include one or more receiving portions, each of which may be adapted to receive a different element or component of the spinal implant. The connector heads 11, 12 may also provide a way of securing the received components within connector head, thus, for example, forming a fixed connection or joint. Exemplary connector heads 11, 12, which are shown as identical in FIGS. 2-3, are described with reference to connector head 12 in FIGS. 2-5. However, this is by way of example only and the connector heads need not all be identical nor include all the features described herein.

As shown in FIGS. 2-5, the connector head 12 generally includes a first receiving portion 27 for receiving the cross member 6 and a second receiving portion 28 for receiving a spinal fixation element, such as the spinal rods 15, 16. In the illustrated embodiment, the connector head 12 has a generally cylindrical shape. However, it may have any shape suitable for implantation in the body. The first receiving portion 27 and the second receiving portion 28 can also represent adjacent portions or sections of the connector head 12. As shown in FIGS. 2-5, the first receiving portion 27 is positioned distal to the second receiving portion 28 and proximal to a closed distal end 34 of the connector head 12. The second receiving portion 28 extends proximally from the first receiving portion 28.

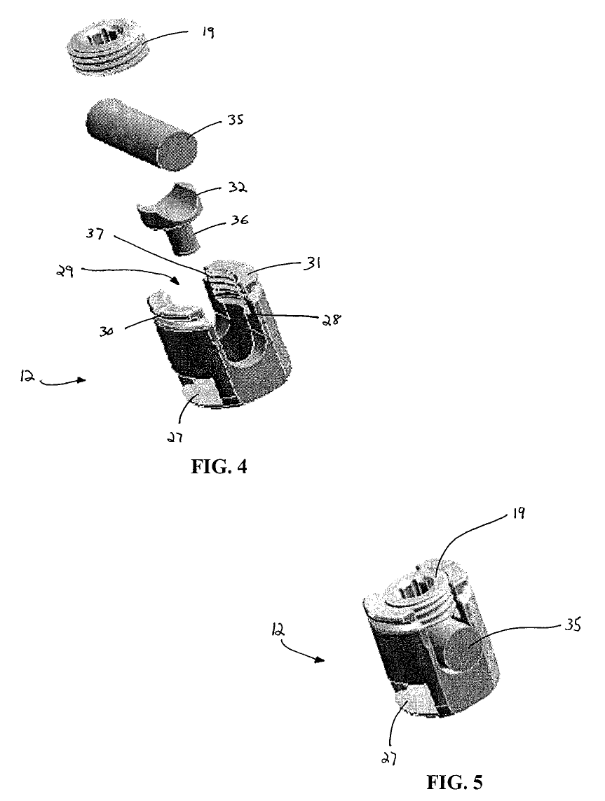

The first receiving portion 27 can have a variety of configurations, but in an exemplary embodiment it is adapted to receive the cross member. In the illustrated embodiment, the first receiving portion 27 is defined by an opening, such as a hole, channel, slot or tunnel, formed through the connector head 12. The cross member 6 can slide through or can be inserted through this opening. In other embodiments, the first receiving portion 27 need not be integral to the connector head 12, but can be defined by an opening created by one or more protruding loops of material or brackets attached to the connector head cylinder 12. FIG. 4 illustrates the connector head 12 in more detail, and shows that the opening of the first receiving portion 27 can be a rectangular hole formed through the lower portion of the connector head 12, and can have a height and width suited for the cross member 6.

Depending on the cross-sectional shape of the cross member 6 and the shape of the opening defining the first receiving portion 27, a surgeon may slide the connector head 12 onto the cross member 6 prior to use. However, the cross member 6 can optionally be fabricated with one or more sliding connector heads 11, 12 pre-assembled on it. In another embodiment, the connector heads 11, 12 can include a generally cylindrical body portion with an open bottom 34, and a separate bottom plate or cap, allowing the surgeon to assemble the connector heads 11, 12 onto the cross member 6.

The second receiving portion 28 of the connector head 12 can also have a variety of configurations, but in an exemplary embodiment it is adapted to receive a spinal fixation element. As shown in FIGS. 2-5, the second receiving portion 28 is defined by a U-shaped recess 29 formed between the opposed arms 30, 31 and configured to accommodate one or more spinal fixation elements, such as the spinal rods 15, 16 as shown in FIG. 1. Although the recess 29 can have any shape, as shown in the figures the recess 29 has substantially straight walls ending in a semi-circular curve along the deepest part of the recess 29. A spinal rod 15, 16 can sit in the U-shaped recess 29. The particular location of the U-shaped recess 29 can also vary. As shown in the figures, the U-shaped recess 29 is positioned offset from the opening that forms the first receiving portion 27. As a result, a spinal fixation element seated within the recess 29 will extend substantially perpendicular to the cross member 6 disposed through the first receiving portion 27. In other embodiments, the U-shaped recess 29 can be aligned with the opening in the first receiving portion 27 such that the spinal fixation element and cross member will extend generally parallel to one another.

As further shown in FIG. 4, the connector head 12 can include a seat 32 disposed therein and configured to seat a spinal fixation element disposed within the connector head 12, to facilitate sliding movement of the connector head 12 along the cross member, and optionally to facilitate locking of the connector head 12 relative to the cross member 6. The configuration of the seat can vary, but in an exemplary embodiment the proximal surface of the seat 32 has a shape that complements the shape of the spinal rod 15, 16 or other spinal fixation element disposed in the recess 29. For instance, as shown in FIGS. 4-5, the seat 32 can is be saddle-shaped to accommodate a cylindrical spinal segment 35. (In FIG. 4, the spinal rod segment 35 simply serves to illustrate how a spinal rod 15, 16 could be used with the connector head 12.) As further shown, the seat 32 can also have a guide pin 36 extending downward from its distal surface to engage or slide in a guide channel 13, 14 (FIG. 2) formed in the cross member 6. In this way, movement of the connector head 12 along the cross member 6 is guided by the guide pin 36. Alternatively a protrusion or tab formed on the connector head 12, for example on the bottom 34 or the interior surfaces 33, can extend into the guide channel 14 in order to guide the connector head 12.

Further optional features of connector head 12 can include a mating element formed thereon for mating with a locking mechanism to lock a spinal fixation element within the connector head 12, and optionally to secure the connector head 12 in place on the cross member 6. By way of non-limiting example, the mating element can be screw threads 37 formed on the lateral interior surfaces 33 of the arms 30, 31. FIG. 4 illustrates the screw threads 37 on the upper area of the arms 30, 31, however they could extend farther down the arms 30, 31 or could be formed on the outside of the arms 30, 31 as well or alternatively. The locking mechanism can be, for example, a set screw 19 adapted to mate with the screw threads 37 and tighten into the connector head 12.

As shown in FIG. 4, a set screw 19 can be placed into the connector head 12 following positioning of a spinal fixation element within the connector head 12. As the set screw 19 tightens, it can bear against the spinal rod 35 placed in the recess 29 causing the spinal rod 35 to bear into the seat 32. This in turn will force the seat 32 against the cross member 6 underneath it thus locking the cross member 6 and connector head 12, thereby preventing sliding movement of the connector head 12 along the cross member 6. FIG. 5 illustrates this assembly (for clarity, the cross member 6 is not shown in FIG. 5 but can reside in the first receiving portion 27 as shown in FIGS. 2-3). Although use of the screw threads 37 and set screw 19 has an advantage of simultaneously securing the spinal rod segment 35 in the connector head 12 and the connector head 12 on the cross member 6, a variety of mating elements and locking mechanisms may be suitable to secure a spinal rod 15, 16 within the connector head 12 and/or to secure the connector head 12 in place on the cross member 6. For example, the seat 32 itself may include a locking mechanism such as a screw, bolt, or the like, that serves to lock the connector head 12 in place before placement of a spinal rod in recess 29. Alternatively, a screw, bolt, bio-compatible cement, or other fastener could be employed from the bottom 34 of the cross member 6 to secure the connector head 12 from the bottom 34. In the case where the guide channel 14 includes teeth or serrations, the insertion of the guide pin 36 can by itself can secure the connector head 12 in place on the cross member 6.

The connector heads 11, 12 need not all be slidably disposed on the cross member 6. One or more connector heads may be fixedly attached to the cross member 6 by welds, bolts, screws, bio-bio-compatible cement, or the like. In addition, the cross member 6 and one or more fixedly attached connector heads can be fabricated as one component, formed of the aforementioned bio-compatible material such as a titanium alloy or stainless steel. In that case, slidably disposed connector heads 11, 12 may be added as required by the particular application, while still retaining the functionality of fixedly attached connector heads.

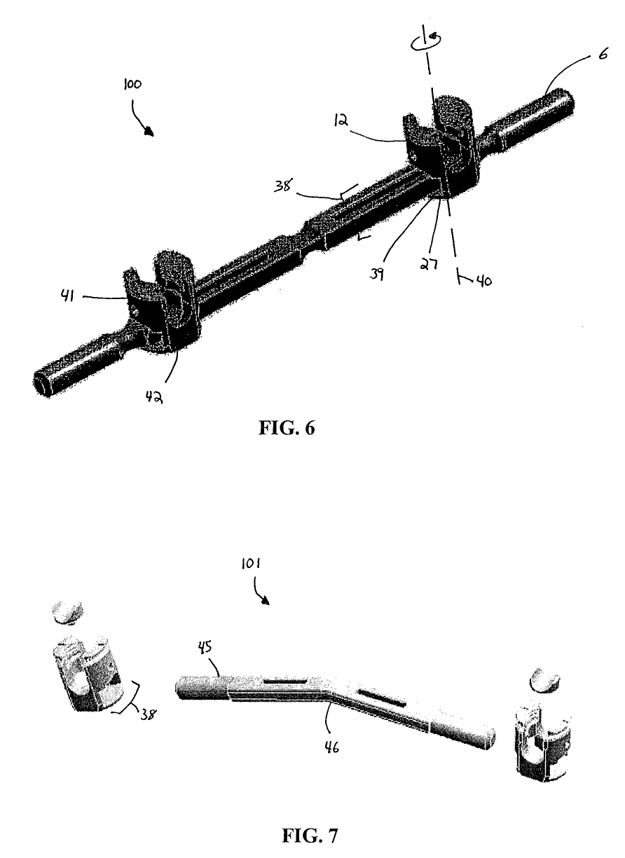

FIG. 6 shows a perspective view of the cross connector 100 of FIGS. 1-3. This view illustrates how the connector head 12 can be adapted to rotate or pivot relative to the cross member 6. The opening defining the first receiving portion 27 of the connector head 12 can be sized such that the width 38 (the width 38 shown clearly in FIG. 7) of the opening is larger than the corresponding width of the cross member 6. The resulting gap 39 between the connector head 12 and the cross member 6 can allow the connector head to pivot relative to the cross member 6. For example, an appropriate gap 39 on either side of cross member 6 can allow the cross member to pivot about axis 40. Moreover, the opening defining the first receiving portion 27 can be sized so that the connector head 12 can pivot over virtually any desired range. It may be useful, for example, to allow the connector head 12 to pivot approximately twenty degrees in either direction relative to the cross member. This pivoting capability provides additional customization options for the spinal rods or other spinal fixation elements connected to the connector head 12. A locking mechanism, as previously described, can be used to secure connector head 12 in place on the cross member 6 and can prevent the connector head 12 from further pivoting.

As an alternative to the pivot created by the gap 39, the connector head 12 can be constructed with an integral pivot, eliminating the need for the gap 39. In such an embodiment, the connector head can include a first body portion 41 with a first receiving portion for receiving the cross member 6, and a second body portion 42 with a second receiving portion for receiving a spinal fixation element. The two body portions can be connected by a pivot, ball joint, or rotating portion defined by ball bearings or a smooth bushing, or the like.

FIG. 7 illustrates an alternative embodiment of a cross connector 101 having a cross member 45. The cross member 45 can optionally include a pre-fabricated anterior angle 46. This embodiment may be particularly suited for sacral fixations, because the angle 46 can accommodate the curvature of the sacrum. Variations of the cross member 45 may be supplied with a range of predetermined angles according to commonly required configurations. In addition, although shown as an anterior angle, the angle 46 could be a posterior, inferior, or superior-facing angle. Although as shown the cross member 45 includes no bend zones, it is also possible that the cross member 45 could include bend zones in order to provide more options for intraoperative customization, or for final adjustment of the angle 46.

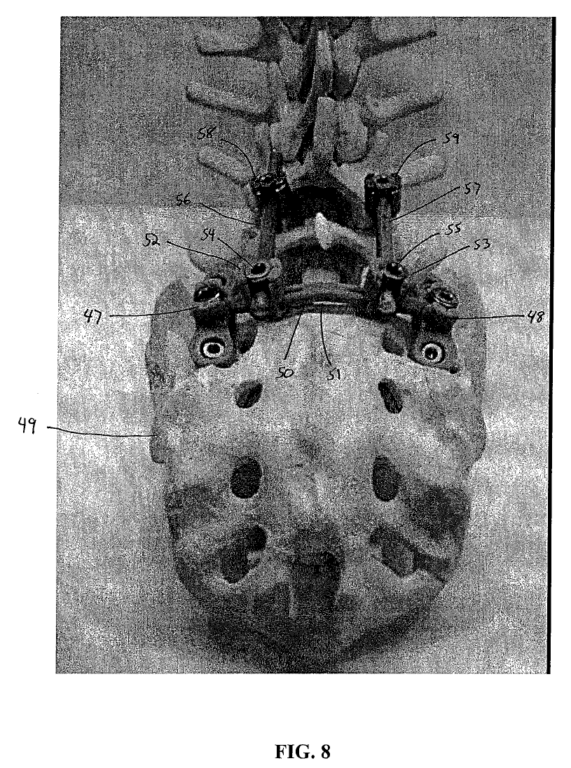

FIGS. 8-11 illustrate various exemplary spinal fixation constructs that utilize a cross connector for anchoring the construct to the sacrum and/or ilium. While the methods and constructs are described in connection with the cross connectors shown in FIGS. 1-7, a person skilled in the art will appreciate that the cross connector can have virtually any configuration, and that the particular configuration can vary depending on the intended use. Moreover, the components used in each construct and the particular configuration of each component can vary. Various other devices known in the art can also be used to provide certain mating connections between the components of the various constructs.

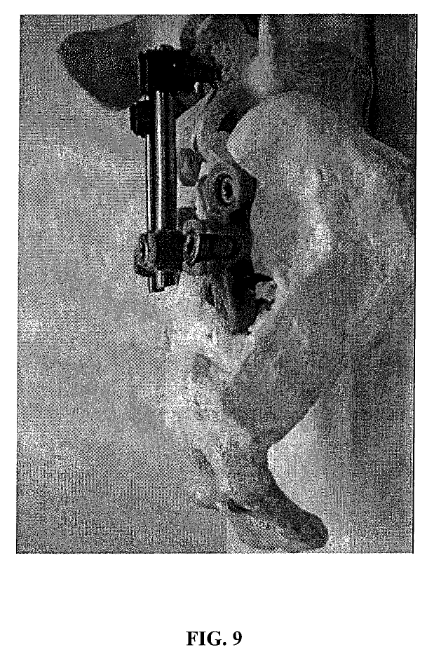

FIGS. 8 and 9 illustrate one exemplary embodiment of a spinal construct for sacral fixation. Referring to FIG. 8, bone anchoring elements, shown as bone plates 47, 48, can be attached to the posterior surface of the sacrum 49. A cross member 50 can have a pre-fabricated anterior angle 51 and can form a transverse connection between the anchoring elements 47, 48. First and second connector heads 52, 53 can be slidably disposed on the cross member 50. The connector heads 52, 52 can be secured in place on the cross member 50 with locking mechanisms 54, 55, shown as set screws. A first spinal rod 56 can sit in a U-shaped recess that defines a second receiving portion of the first connector head 52, and it can extend from the connector head 52 to the anchoring element 58. Likewise, a second spinal rod 57 can sit in a U-shaped recess that defines a second receiving portion of connector head 53, and it can extend from the connector head 53 to the anchoring element 59. The anchoring elements 58, 59 are shown attached to the fourth lumbar vertebra. FIG. 9 is a side view of the spinal construct configured for sacral fixation of FIG. 8.

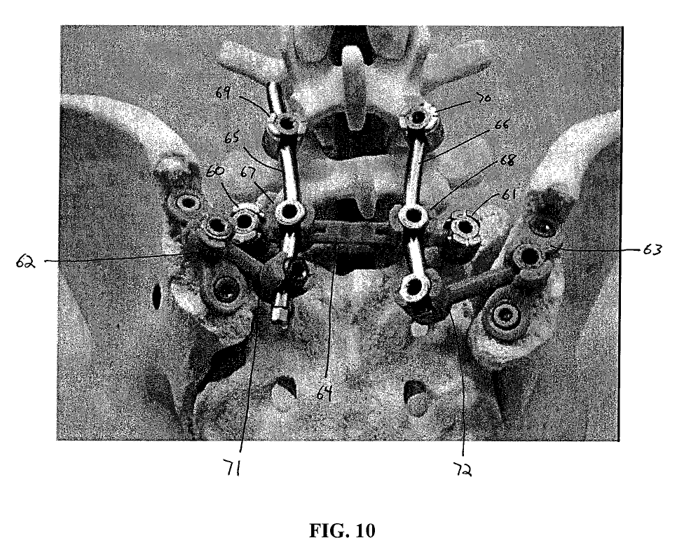

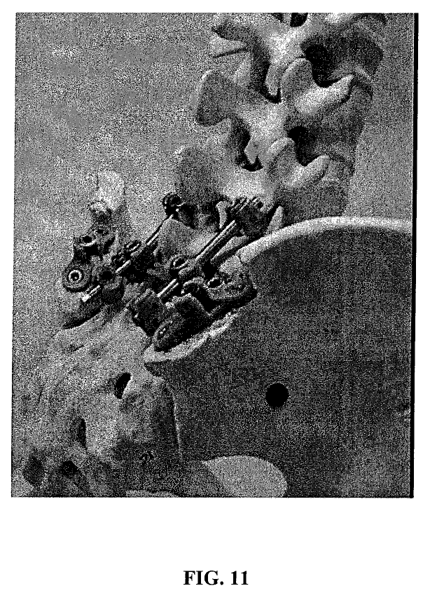

FIGS. 10 and 11 illustrate another exemplary embodiment of a spinal construct configured for sacroiliac fixation. The bone anchoring elements 60, 61 can be placed on the sacrum, while the anchoring elements 62, 63 can reside on the ilium. The cross member 64 can form a transverse connection between the anchoring elements 60, 61. A first spinal rod 65 can be disposed in the recess of the first connector head 67, which can be secured into place on the cross member 64. A second spinal rod 66 can be disposed in the recess of the second connector head 68, which can also be secured into place on the cross member 64. The first spinal rod 65 can extend longitudinally along the spine from the anchoring element 69, which as shown is attached to the inferior articular process of the fourth lumbar vertebra, to the articulating spinal fixation element 71. The second spinal rod 66 can extend longitudinally along the spine from the anchoring element 70, which as shown is also attached to the inferior articular process of the fourth lumbar vertebra, to the articulating spinal fixation element 72. The articulating spinal fixation elements 71, 72 shown herein are described in more detail in a U.S. patent application filed on even date herewith and entitled “Articulating Sacral or Iliac Connector,” by Nam T. Chao et al. (Attorney Docket No. 101896-470), which is hereby incorporated by reference in its entirety.

The connector heads 67, 68 are shown positioned as far apart as possible on cross member 64. The spinal rods 65, 66 can therefore remain relatively straight in forming the connections between anchoring elements on the fourth lumbar vertebra and the articulating spinal fixation elements 71, 72. Accordingly, FIG. 11 illustrates one way in which the cross member 64 and the slidably disposed connector heads 67, 68 can reduce the extent to which a surgeon must contour the spinal rods 65, 66. FIG. 11 is a side view of the spinal construct configured for sacroiliac fixation of FIG. 10.

One exemplary method for correcting spinal deformities is described by reference to the construct of FIG. 1. However, one skilled in the art will recognize that the described method is not at all limited to the construct shown in FIG. 1 or to the cross connectors described by reference to FIGS. 1-12 above. The particular configuration of the construct, the components employed therein, and the features of the components themselves can vary depending on the intended use. Turning to FIG. 1, a surgeon can anchor the cross connector 100 to bone, for example on the sacrum or ilium, using the anchoring elements 8, 10. As will be understood by those of skill in the art, virtually any location is possible and can be suitable for the surgical technique of the surgeon, who will commonly desire a particular fixation based on patient anatomy and the intended corrective effect. The location chosen may require that the surgeon prepare the bone to accommodate the anchoring element. In FIG. 1, the selected anchor locations are on the left ilium 2 and right ilium 4. One approach can involve securing the anchoring elements to bone with bone screws, inserting a first end 7 of the cross member 6 into anchoring element 8, then inserting the opposing second end 9 of the cross member 6 into anchoring element 10. The anchors 17, 18 can be placed on the lumbar vertebra, or other suitable location. The surgeon can connect the anchor 17 and the first connector head 11 with a first longitudinal spinal rod 15, and connect the anchor 18 and the second connector head 12 with a second longitudinal spinal rod 16. The spinal rods 15, 16 may require iterative contouring in order to achieve the desired corrective effect. However, the surgeon can also laterally slide and/or rotate connector heads 11, 12 to facilitate placement of the spinal rods 15, 16, respectively. Once the connector head 11 is in a desired position, the surgeon can lock the connector head 11 in place on the cross member 6 and simultaneously secure the spinal rod 15 in the connector head 11 with the locking mechanism 19, which as shown can be an interior set screw that the surgeon tightens. The other connector head 12 can be likewise locked into position and the spinal rod 16 secured in the connector head with locking mechanism 20.

One skilled in the art will appreciate further features and advantages of the invention based on the above-described embodiments. Accordingly, the invention is not to be limited by what has been particularly shown and described, except as indicated by the appended claims. All publications and references cited herein are expressly incorporated herein by reference in their entirety.

What is claimed is:

1. An implantable device for connecting a spinal fixation element to a patient's sacrum or ilium, comprising:
a cross member having opposed first and second ends, and a guide channel formed between the first and second ends; and
a connector head slidably disposed on the guide channel, the connector head including a first receiving portion defined by an opening through the connector head for receiving the cross member, and a second receiving portion defined by a recess formed between first and second opposed arms for receiving a spinal fixation element.

2. The device of claim 1, further comprising a guide pin extending from the connector head into the guide channel for guiding movement of the connector head along the cross member.

3. The device of claim 1, further comprising a seat disposed in the recess of the second receiving portion of the connector head for receiving a spinal fixation element.

4. The device of claim 3, further comprising a guide pin extending from the seat into the guide channel for guiding movement of the connector head along the cross member.

5. The device of claim 3, wherein the seat is saddle-shaped.

6. The device of claim 1, further comprising a locking mechanism adapted to mate to the connector head to lock a spinal fixation element therein and to lock the connector head in a fixed position relative to the cross member.

7. The device of claim 6, wherein the connector head includes screw threads formed thereon for mating with a locking mechanism.

8. The device of claim 1, wherein the opening that defines the first receiving portion of the connector head has a width that exceeds a width of the cross member such that the connector head can pivot relative to the cross member.

9. The device of claim 1, further comprising a second connector head fixedly attached to the cross member.

10. The device of claim 1, further comprising a second connector head slidably disposed on the cross member and including a first receiving portion defined by an opening through the connector head for receiving the cross member, and a second receiving portion defined by a recess formed between first and second opposed arms for receiving a spinal fixation element.

11. The device of claim 10, further comprising a second guide channel provided on the cross member between the first and second ends, the second connector head being slidably disposed on the second guide channel.

12. The device of claim 11, further comprising a second locking mechanism adapted to mate to the second connector head to lock a spinal fixation element therein and thereby lock the second connector head in a fixed position relative to the cross member.

13. The device of claim 11, further comprising a guide pin extending from the second connector head into the second guide channel for guiding movement of the second connector head along the cross member.

14. The device of claim 1, wherein the cross member has at least one bend zone.

15. A system for connecting a spinal fixation element to a patient's sacrum or ilium, comprising:
a cross member having opposed first and second ends;
a spinal fixation element;
a connector head slidably disposed on the cross member between the first and second ends and adapted to receive the spinal fixation element; and
a locking mechanism for locking the spinal fixation element within the connector head to prevent movement of the connector head relative to the cross member.

16. The system of claim 15, wherein the connector head includes a first receiving portion defined by an opening through the connector head for receiving the cross member, and a second receiving portion defined by a recess between first and second opposed arms for receiving a spinal fixation element.

17. The system of claim 16, further comprising a guide channel on the cross member between the first and second ends, the connector head being slidably disposed on the guide channel.

18. The system of claim 17, further comprising a seat disposed in the recess of the second receiving portion of the connector head for receiving the spinal fixation element.

19. The system of claim 18, further comprising a guide pin extending from the seat into the guide channel for guiding the movement of the connector head along the cross member.

20. The system of claim 16, wherein the opening that defines the first receiving portion of the connector head has a width that exceeds a width of the cross member such that the connector head can pivot relative to the cross member.

21. The system of claim 15, further comprising at least one anchoring element connected to at least one of the first and second ends of the cross member for anchoring the cross member to bone.

22. The system of claim 15, further comprising a second spinal fixation element, and a second connector head disposed on the cross member between the first and second ends and adapted to receive the second spinal fixation element.

23. The system of claim 22, wherein the second connector head is slidably disposed on the cross member.

24. The system of claim 22, wherein the second connector head is fixedly attached to the cross member.

25. The system of claim 15, wherein the cross member has at least one bend zone.

26. A method for anchoring a spinal fixation element to a patient's sacrum or ilium, comprising:
anchoring a cross member to bone;
sliding a connector head along the cross member;
positioning a spinal fixation element within the connector head; and
locking the spinal fixation element within the connector head to thereby lock the connector head in a fixed position relative to the cross member.

27. The method of claim 26, further comprising positioning a second spinal fixation element within a second connector disposed on the cross member.

28. The method of claim 27, further comprising sliding the second connector along the cross member to facilitate positioning of the second spinal fixation element therein, and locking the second spinal fixation element within the second connector head to thereby lock the second connector head in a fixed position relative to the cross member.

29. The method of claim 28, wherein the connector head is slid along a first guide channel formed in the cross member, and the second connector head is slid along a second guide channel formed in the cross member.

30. The method of claim 26, further comprising bending the cross member at a bend zone.

31. The method of claim 26, further comprising pivoting the connector head relative to the cross member.
